(19) United States
(12) Patent Application Publication
(10) Pub. No.: US 2009/0018051 A1
(43) Pub. Date: Jan. 15, 2009

(54) USES OF (Z)-1-(3-METHYL-BUT-2-ENYLOXY)-HEX-3-ENE

(75) Inventors: Aurelia Reckziegel, Dormagen (DE); Horst Surburg, Holzminden (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzminden (DE)

(21) Appl. No.: 11/997,986

(22) PCT Filed: Aug. 4, 2006
(86) PCT No.: PCT/EP2006/065088

§ 371 Date: Aug. 27, 2008

(30) Foreign Application Priority Data
Aug. 6, 2005 (DE) 10 2005 037 184.1

(51) Int. Cl. A61K 8/33; A61Q 13/00

(52) U.S. Cl. 512/26

(57) ABSTRACT

The use of (Z)-1-(3-methylbut-2-enyloxy)hex-3-ene, or a mixture containing (Z)-1-(3-methylbut-2-enyloxy)hex-3-ene, as an agent for providing (a) hair or (b) textile fibres with a fresh-green smell is described.

The invention relates to novel uses of (Z)-1-(3-methylbut-2-enyloxy)hex-3-ene (cis-3-hexenyl prenyl ether) and mixtures containing this compound.

In Pishchevaya Promyshlennost (Moscow, Russian Federation) (1990), (1), 62-63, various (Z)-hex-3-enyl ethers are prepared from (Z)-hex-3-en-1-ol (leaf alcohol) and described in olfactory terms. They include (Z)-1-(3-methylbut-2-enyloxy)hex-3-ene, whose odour is described as flowery-fruity with green nuances. It is also mentioned that the disclosed (Z)-hex-3-enyl ethers can be used in perfumery.

In our own experiments (Z)-1-(3-methylbut-2-enyloxy)hex-3-ene exhibited a very intense, extremely natural and modern green odour coupled with flowery-fruity notes. The green odour is reminiscent of peas, green beans and leaves, the fruity note is determined by apple and the flowery note by violet, and herbal nuances are also evident. Overall, the olfactory properties of (Z)-1-(3-methylbut-2-enyloxy)hex-3-ene resemble those of the commercially available fragrances (Z)-1-(1-ethoxyethoxy)hex-3-ene (CAS No. 28069-74-1 leaf acetal, product name of IFF) and 4-methyl-3-decen-5-ol (CAS No. 81782-77-6, undecavertol, product name of Givaudan).

U.S. Pat. No. 6,340,666 describes 3-hexenyl 2-methylallyl ether in olfactory terms as a compound with fresh notes such as green, mushroom-like, freshly cut grass, fruity (rhubarb) and violet. It is argued, without detailed justification, that the (Z) isomer has the more attractive olfactory properties. It is also mentioned that this compound can be used not only in fine perfumery but also e.g. for the perfuming of soaps, cleaning agents and detergents or fabric softeners.

Fragrance mixtures with a particularly fresh-green top note, which simultaneously have a pronounced blooming (odour from an aqueous surfactant solution), are often sought after, especially for the perfuming of formulations containing surfactants, such as shampoos, detergents or fabric softeners. Another important application technology requirement of fragrance mixtures for products containing surfactants is their substantivity towards or retention on the substrate, especially hair or textile fibres. The meaning of substantivity and retention is explained in detail e.g. in EP 1 201 738 A1 cf. sections [0004]-[0005]. In general, therefore, fragrances with a high substantivity and/or retention are also sought after.

A first feature of the present invention, which is closely connected with the general objects mentioned above, relates to the use of (Z)-1-(3-methylbut-2-enyloxy)hex-3-ene, or a mixture containing (Z)-1-(3-methylbut-2-enyloxy)hex-3-ene, as an agent for providing (a) hair or (b) textile fibres with a fresh-green smell.

Pishchevaya Promyshlennost (Moscow, Russian Federation) (1990), (1), 62-3, makes no reference to the particularly high substantivity, retention and fixing action of (Z)-1-(3-methylbut-2-enyloxy)hex-3-ene, which is surprising in comparison with structurally related substances and is responsible for the fact that the use according to the invention produces outstanding results. In fact, hair and textile fibres which have been treated with (Z)-1-(3-methylbut-2-enyloxy)hex-3-ene have a fresh, green, flowery-fruity smell that persists for a surprisingly long time and is also particularly resistant to washing (with water).

In accordance with these findings, a further feature of the invention relates to the use of (Z)-1-(3-methylbut-2-enyloxy)hex-3-ene as an agent for increasing the substantivity and/or retention of a fragrance mixture (towards or on hair or textile fibres). By adding (Z)-1-(3-methylbut-2-enyloxy)hex-3-ene to a given fragrance mixture of only low substantivity and/or retention, these properties are improved in a particularly advantageous manner. Thus, for example, by the addition of (Z)-1-(3-methylbut-2-enyloxy)hex-3-ene, an aqueous washing solution (or a corresponding detergent or shampoo or the like) that smells fresh but, because of the inadequate substantivity of the perfumes it contains, is not suitable for passing on a fresh odour to fabrics (textile fibres) or hair can be converted to a solution that is outstanding at passing on a fresh odour which persists for a long time on the treated substrates (hair or textile fibres).

A corresponding process according to the invention for providing (a) hair or (b) textile fibres with a fresh smell comprises the following steps.
preparation of a mixture containing (Z)-1-(3-methylbut-2-enyloxy)hex-3-ene, and
application of the mixture to the hair or textile fibres.

A mixture which is particularly suitable for the uses according to the invention or the corresponding processes is a solution comprising (a) water, (b) (Z)-1-(3-methylbut-2-enyloxy)hex-3-ene and (c) one or more surfactants, the concentration of (Z)-1-(3-methylbut-2-enyloxy)hex-3-ene in the solution being in the range from 10⁻⁷ to 10⁻¹ wt. %. Other fragrances and/or miscellaneous conventional additives can be present.

The already mentioned in Pishchevaya Promyshlennost (Moscow, Russian Federation) (1990), (1), 62-3, makes no reference to the particular olfactory effects of (Z)-1-(3-methylbut-2-enyloxy)hex-3-ene in the context of aqueous products containing surfactants.

Surprisingly, in the uses, processes, solutions and mixtures according to the invention, the use of (Z)-1-(3-methylbut-2-enyloxy)hex-3-ene achieves a natural, modern, very strong green top note (including interesting violet and apple nuances) not only in conjunction with a high substantivity/retention but also in conjunction with a surprising blooming (i.e. the odour perceived above an aqueous solution containing surfactants). This combination of sought-after properties could not be found in the state of the art.

As the Examples below show in detail, particularly the compound (Z)-1-(1-ethoxyethoxy)hex-3-ene (leaf acetal, product name of IFF), which is very similar in olfactory terms to (Z)-1-(3-methylbut-2-enyloxy)hex-3-ene, does not exhibit this fortunate combination of properties.

Accordingly, a further feature of the invention relates to the use of (Z)-1-(3-methylbut-2-enyloxy)hex-3-ene for increasing the odour of other fragrances perceived above an aqueous solution containing surfactants (e.g. a wash liquor), i.e. for increasing the blooming.

The desired fresh top note with a pronounced blooming and an increased substantivity for aqueous applications involving surfactants can normally be achieved by using only a small dose of (Z)-1-(3-methylbut-2-enyloxy)hex-3-ene in a resulting perfume composition.

(Z)-1-(3-methylbut-2-enyloxy)hex-3-ene can be used in a large number of products. The pH of an aqueous formulation containing the ether to be used according to the invention is not critical because (Z)-1-(3-methylbut-2-enyloxy)hex-3-ene is very stable in both acidic and basic media (cf. Examples 1-3 in this respect). It has been found, however, that the ether to be used according to the invention exhibits and effects a particularly pronounced blooming in applications/formulations of basic pH, so the pH is preferably in the range from 8 to 13. The pH of a solution according to the invention is preferably adjusted to this range.

Examples of fragrances with which (Z)-1-(3-methylbut-2-enyloxy)hex-3-ene can advantageously be combined can be found e.g. in S. Arctander, Perfume and Flavor Materials, Vol. I and II, Montclair, N.J., 1969, Selbstverlag, or K. Bauer, D. Garbe and H. Surburg, Common Fragrance and Flavor Materials, 4th Ed., Wiley-VCH, Weinheim 2001.

The following may be specifically mentioned:

extracts of natural raw materials, such as ethereal oils, concrete oils, absolute essences, resins, resinoids, balsams, tinctures, e.g.:
ambergris tincture; amyris oil; angelica seed oil; angelica root oil; anise oil; baldrian oil; basil oil; tree moss absolute; bay oil; mugwort oil; benzoin resin; bergamot oil; beeswax absolute; birch tar oil; bitter almond oil; savory oil; buchu leaf oil; cabreuva oil; cade oil; calmus oil; camphor oil; cananga oil; cardamom oil; cascarilla oil; cassia oil; cassia absolute; castoreum absolute; cedar leaf oil; cedarwood oil; cistus oil; citronella oil; lemon oil; copaiva balsam; copaiva balsam oil; coriander oil; costus root oil; cumin oil; cypress oil; davana oil; dill oil; dill seed oil; eau de brouts absolute; oak moss absolute; elemi oil; tarragon oil; eucalyptus citriodora oil; eucalyptus oil; fennel oil; pine needle oil; galbanum oil; galbanum resin; geranium oil; grapefruit oil; guaiacum wood oil; gurjun balsam; gurjun balsam oil; helichrysum absolute; helichrysum oil; ginger oil; iris root absolute; iris root oil; jasmine absolute; calamus oil; blue camomile oil; Roman camomile oil; carrot seed oil; cascarilla oil; Scotch fir oil; spearmint oil; caraway oil; ladanum oil; ladanum absolute; ladanum resin; lavandin absolute; lavandin oil; lavender absolute; lavender oil; lemongrass oil; lovage oil; distilled lime oil; pressed lime oil; linaloa oil; litsea cubeba oil; laurel leaf oil; mace oil; marjoram oil; mandarin oil; massoi bark oil; mimosa absolute; ambrette oil; musk tincture; muscatel sage oil; nutmeg oil; myrrh absolute; myrrh oil; myrtle oil; clove leaf oil; clove blossom oil; neroli oil; frankincense absolute; frankincense oil; opopanax oil; orange blossom absolute; orange oil; oreganum oil; palmarosa oil; patchouli oil; perilla oil; Peruvian balsam oil; parsley leaf oil; parsley seed oil; petitgrain oil; peppermint oil; pepper oil; pimento oil; Pinus pinea oil; European pennyroyal oil; rose absolute; rosewood oil; rose oil; rosemary oil; Dalmatian sage oil; Spanish sage oil; sandalwood oil; celery seed oil; lavender spike oil; Japanese anise oil; styrax oil; tagetes oil; fir needle oil; tea tree oil; turpentine oil; thymian oil; tolu balsam; tonka absolute; tuberose absolute; vanilla extract; violet leaf absolute; verbena oil; vetiver oil; juniper oil; wine yeast oil; wormwood oil; wintergreen oil; ylang oil; hyssop oil; civet absolute; cinnamon leaf oil; cinnamon bark oil; and fractions thereof or ingredients isolated therefrom; individual fragrances from the following groups:
hydrocarbons, e.g. 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;
aliphatic alcohols, e.g. hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
aliphatic aldehydes and their acetals, e.g. hexanal; heptanal; octanal; nonanal; decanat; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanal diethyl acetal; is 1,1-dimethoxy-2,2,5-trimethyl-4-hexene; citronellyloxyacetaldehyde;
aliphatic ketones and their oximes, e.g. 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
aliphatic sulfur-containing compounds, e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
aliphatic nitrites, e.g. 2-nonenoic acid nitrile; 2-tridecenoic acid nitrile; 2,12-tridecadienoic acid nitrile; 3,7-dimethyl-2,6-octadienoic acid nitrile; 3,7-dimethyl-6-octenoic acid nitrile;
aliphatic carboxylic acids and their esters, e.g. (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octynate; methyl 2-nonynate; allyl 2-isoamyloxyacetate; methyl 3,7-dimethyl-2,6-octadienoate;
acyclic terpene alcohols, e.g. citronellol; geraniol; nerol; linalool; lavadulol; nerolidol; farnesol; tetrahydrolinalool; tetrahydrogeraniol; 2,6-dimethyl-7-octen-2-ol; 2,6-dimethyloctan-2-ol; 2-methyl-6-methylen-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and their formates, acetates, propionates, isobutyrates, butyrates, isovalerates, pentanoates, hexanoates, crotonates, tiglates, 3-methyl-2-butenoates;
acyclic terpene aldehydes and ketones, e.g. geranial; neral; citronellal; 7-hydroxy-3,7-dimethyloctanal; 7-methoxy-3,7-dimethyloctanal; 2,6,10-trimethyl-9-undecenal; geranylacetone; and the dimethyl and diethyl acetals of geranial, neral, 7-hydroxy-3,7-dimethyloctanal;
cyclic terpene alcohols, e.g. menthol; isopulegol; alpha-terpineol; terpinen-4-ol; menthan-8-ol; menthan-1-ol; menthan-7-ol; borneol; isoborneol; linalool oxide; nopol; cedrol; ambrinol; vetiverol; guaiol; and their formates, acetates, propionates, isobutyrates, butyrates, isovalerates, pentanoates, hexanoates, crotonates, tiglates, 3-methyl-2-butenoates;
cyclic terpene aldehydes and ketones, e.g. menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4-a-methanonaphthalen-8(5H)-one; nootkatone; dihydronootkatone; alpha-sinensal; beta-sinensal; acetylated cedarwood oil (methyl cedryl ketone); cyclic alcohols, e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
cycloaliphatic alcohols, e.g. alpha-3,3-trimethylcyclohexylmethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
cyclic and cycloaliphatic ethers, e.g. cineol; cedryl methyl ether; cyclododecyl methyl ether; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
cyclic and macrocyclic ketones, e.g. 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopenta-decanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4-(5H)-indanone; 8-cyclohexadecen-1-one; 9-cycloheptadecen-1-one; cyclopentadecanone; cyclohexadecanone;
cycloaliphatic aldehydes, e.g. 2,4-dimethyl-3-cyclohexenecarbaldehyde; 2-methyl-4-(2,2,6-trimethylcyclohexen-1-yl)-2-butenal; 4-(4-hydroxy-4-methyl-pentyl)-3-cyclohexenecarbaldehyde; 4-(4-methyl-3-penten-1-yl)-3-cyclohexenecarbaldehyde; cycloaliphatic ketones, e.g. 1-(3,3-dimethylcyclohexyl)-4-penten-1-one; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl 2,4-dimethyl-3-cyclohexen-1-yl ketone; esters of cyclic alcohols, e.g. 2-tert-butylcyclohexyl acetate; 4-tert-butylcyclohexyl acetate; 2-tert-pentylcyclohexyl acetate; 4-tert-pentylcyclohexyl acetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or 6-indenyl acetate; 4,7-methano-3a,4,5,6,7,7a-hexahydro-5- or 6-indenyl propionate; 4,7-methano-3a,4,5,6,7,7a-hexahydro-5- or 6-indenyl isobutyrate; 4,7-methanooctahydro-5- or 6-indenyl acetate;
esters of cycloaliphatic carboxylic acids, e.g. allyl 3-cyclohexylpropionate; allyl cyclohexyloxyacetate; cis- and trans-methyl dihydrojasmonate; cis- and trans-methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexene-carboxylate; ethyl 2-methyl-1,3-dioxolan-2-acetate; araliphatic alcohols, e.g. benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenyl-propanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
esters of araliphatic alcohols and aliphatic carboxylic acids, e.g. benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethyl-phenylethyl acetate; alpha,alpha-dimethylphenylethyl butyrate; cinnamyl acetate; 2-phenoxyethyl isobutyrate; 4-methoxybenzyl acetate;
araliphatic ethers, e.g. 2-phenylethyl methyl ether; 2-phenylethyl isoamyl ether; 2-phenylethyl 1-ethoxyethyl ether; phenylacetaldehyde dimethyl acetal; phenyl-acetaldehyde diethyl acetal; hydratropaldehyde dimethyl acetal; phenylacetaldehyde glyceryl acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;
aromatic and araliphatic aldehydes, e.g. benzaldehyde; phenylacetaldehyde; 3-phenylpropanal; hydratropaldehyde; 4-methylbenzaldehyde; 4-methylphenyl-acetaldehyde; 3-(4-ethylphenyl)-2,2-dimethylpropanal; 2-methyl-3-(4-isopropylphenyl)propanal; 2-methyl-3-(4-tert-butylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
aromatic and araliphatic ketones, e.g. acetophenone; 4-methylacetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′, 8′-tetrahydro-3′,5′,5′,6′,8′,8′-hexamethyl-2-acetonaphthone;
aromatic and araliphatic carboxylic acids and their esters, e.g. benzoic acid; phenylacetic acid; methyl benzoate; ethyl benzoate; hexyl benzoate; benzyl benzoate; methylphenyl acetate; ethylphenyl acetate; geranylphenyl acetate; phenylethylphenyl acetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate, benzyl salicylate; phenylethyl salicylate; methyl 2,4-dihydroxy-3,6-dimethylbenzoate; ethyl 3-phenylglycidate; ethyl 3-methyl-3-phenylglycidate;
aromatic nitrogen-containing compounds, e.g. 2,4,6-trinitro-1,3-dimethyl-5-tert-butylbenzene; 3,5-dinitro-2,6-dimethyl-4-tert-butylacetophenone; cinnamic acid nitrile; 5-phenyl-3-methyl-2-pentenoic acid nitrile; 5-phenyl-3-methylpentanoic acid nitrile; methyl anthranilate; methyl N-methylanthranilate; Schiff bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropyl-quinoline; 6-isobutylquinoline; 6-sec-butylquinoline; indole; skatole; 2-methoxy-3-isopropylpyrazine; 2-isobutyl-3-methoxypyrazine;
phenols, phenyl ethers and phenyl esters, e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresylphenyl acetate;
heterocyclic compounds, e.g. 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
lactones, e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 1,15-pentadecanolide; cis- and trans-11-pentadecen-1,15-olide; cis- and trans-12-pentadecen-1,15-olide; 1716-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

Perfume compositions are preferably prepared using combinations with macrocyciic musk fragrances, e.g. 1,15-pentadecanolide, cis- and trans-11-pentadecen-1,15-olide, cis- and trans-12-pentadecen-1,15-olide, 1,16-hexadecanolide, 9-hexadecen-1,16-olide, 10-oxa-1,16-hexadecanolide, 11-oxa-1,16-hexadecanolide, 12-oxa-1,16-hexadecanolide, ethylene 1,12-dodecanedioate and ethylene 1,13-tridecanedioate.

The amount of (Z)-1-(3-methylbut-2-enyloxy)hex-3-ene used in perfume compositions is 0.05 to 50 wt. %, preferably 0.5 to 20%, based on the total perfume composition.

Perfume oils containing (Z)-1-(3-methylbut-2-enyloxy)hex-3-ene can be used in concentrated form, in solutions or in the modified forms described below for the perfuming of e.g. acidic, alkaline and neutral cleaning agents such as carpet cleaning powders and foams, liquid detergents, powder detergents, fabric preconditioners like bleach, soaker and stain remover, fabric softeners, washing soaps, washing tablets, body care products such as solid and liquid soaps, shower gels, shampoos, cosmetic emulsions of the oil-in-water, water-in-oil and water-in-oil-in-water type, and hair care products such as hair sprays, hair gels, strengthening hair lotions, hair rinses, permanent and semipermanent hair dyes, hair styling products like cold waving and straightening products, hair tonics, hair creams and hair lotions.

Perfume oils containing the ether to be used according to the invention can be used in perfumed products in liquid form, either undiluted or diluted with a solvent. Examples of suitable solvents for this purpose are ethanol, isopropanol, diethylene glycol monoethyl ether, glycerol, propylene glycol, 1,2-butylene glycol, dipropylene glycol, diethyl phthalate, triethyl citrate, isopropyl myristate, etc.

Perfume oils containing the ether to be used according to the invention can also be adsorbed on a carrier to ensure both a fine distribution of the fragrances in the product and a controlled release when applied. Such carriers can be porous inorganic materials such as light sulfate, silica gels, zeolites, gypsums, clays, clay granules, gas concrete, etc., or organic materials such as woods and cellulose-based substances.

Perfume oils containing the ether to be used according to the invention can also be microencapsulated or spray-dried or in the form of inclusion complexes or extrusion products, and can be added in this form to the product to be perfumed.

Optionally, the properties of the perfume oils modified in this way can be further optimized, in respect of a more specific perfume release, by coating with suitable materials; waxy plastics, e.g. polyvinyl alcohol, are preferably used for this purpose.

Microencapsulation of the perfume oils can be effected for example by the so-called coacervation process with the aid of capsule materials made e.g. of polyurethane-like substances or soft gelatin. Spray-dried perfume oils can be prepared for example by spray drying an emulsion or dispersion containing the perfume oil, it being possible for modified starches, proteins, dextrins and vegetable gums to be used as carriers. Inclusion complexes can be prepared for example by introducing dispersions of the perfume oil and cyclodextrins or urea derivatives into a suitable solvent, e.g. water. Extrusion products can be prepared by melting the perfume oils with a suitable waxy substance and by extrusion with subsequent solidification, optionally in a suitable solvent, e.g. isopropanol.

Preferred products which can be used within the framework of the present invention are (a) perfume oil mixtures for formulations containing surfactants, e.g. cleaning agents, detergents, fabric softeners and body care products, and (b) the corresponding formulations themselves which contain surfactants.

The formulations containing surfactants which can be used within the framework of the present invention generally include substances from the class of anionic surfactants, e.g. carboxylates, sulfates, sulfonates and phosphates, cationic surfactants, e.g. quaternary ammonium salts, amphoteric surfactants, e.g. betaines, and non-ionic surfactants, e.g. ethoxylates and propoxylates.

Preferred anionic surfactants are sulfates and sulfonates. Preferred sulfates are those having 12 to 18 carbon atoms and a degree of ethoxylation of 1 to at most 5. Sodium laurylethersulfate, preferably having a mean degree of ethoxylation of 2 to 4, is particularly preferred.

Particularly preferred sulfonates are linear sodium alkylbenzenesulfonates having an average of approx. 12 carbon atoms in the alkyl chain, said alkyl chains consisting of homologous radicals having 10 to 14 carbon atoms (“dodecylbenzenesulfonate”).

Preferred compounds from the group of non-ionic surfactants are ethoxylated fatty alcohols obtained by the ethoxylation of alcohols having 12 to 18 carbon atoms (fatty alcohol ethoxylates having 12 to 18 C atoms). The degree of ethoxylation here can vary within wide limits, but particularly preferred products are those having an average degree of ethoxylation of 5 to 10 or, in particular, 7 mol of added ethylene oxide per mol of fatty alcohol.

Particularly preferred betaines are those of the acid amide type having the structure shown:

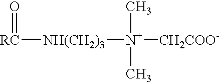

A preferred radical RC═O is the coconut oil fatty acid cut in which lauric acid is the main constituent at 45-50%.

In combination with selected surfactants, the favourable properties of (Z)-1-(3-methylbut-2-enyloxy)hex-3-ene are surprisingly pronounced. A corresponding surfactant formulation according to the invention (mixture according to the invention) comprises (Z)-1-(3-methylbut-2-enyloxy)hex-3-ene and one or more surfactants selected from the group comprising:
linear alkylbenzenesulfonates (especially those mentioned above, e.g. linear sodium alkylbenzenesulfonates),
fatty alcohol ethoxylates having 12-18 C atoms (especially those mentioned above, e.g. those having the degree of ethoxylation identified above as preferred),
laurylethersulfates (especially those mentioned above, e.g. the sodium laurylethersulfate mentioned above) and
betaines (especially those mentioned above, e.g. betaines of the acid amide type having the structure shown above).

Linear alkylbenzenesulfonates and fatty alcohol ethoxylates having 12-18 C atoms are preferably used together with one another here, especially in heavy-duty detergent powders.

Likewise, laurylethersulfates (especially the sodium laurylethersulfate mentioned above) and betaines (especially those of the acid amide type having the structure shown above) are preferably used together with one another, especially in light-duty detergents, shampoos and shower gels.

The concentration of surface-active substances in the surfactant formulations according to the invention is not normally critical. Preferred concentrations depend on the type of surfactant and the particular application. For example, they can be less than 1 wt. % in special bleach products, but greater than 99 wt. % in soaps or washing powder.

Particular combinations and concentrations are preferred in surfactant formulations according to the invention for particular fields of application. Thus, preferred mixtures according to the invention (detergent formulations) are those in which the proportion of linear alkylbenzenesulfonates is in the range from 7 to 10 wt. % and/or the proportion of fatty alcohol ethoxylates having 12-18 C atoms is in the range from 3 to 6 wt. %, based in each case on the total weight of the mixture. Other preferred mixtures according to the invention (formulations for light-duty detergents, shampoos and shower gels) are those in which the proportion of sodium laurylethersulfate is in the range from 7 to 13 wt. % and/or the proportion of betaine (especially betaine of the acid amide type having the structure shown above) is in the range from 1 to 3 wt. %, based in each case on the total weight of the mixture.

When surfactant formulations according to the invention are applied, the substantivity of the (Z)-1-(3-methylbut-2-enyloxy)hex-3-ene on hair and textile fibres is so pronounced as to give the impression that although the surfactants present initially bring the (Z)-1-(3-methylbut-2-enyloxy)hex-3-ene into the aqueous phase, this compound, in the presence of hair or textile fibres, is forced out of the aqueous phase and onto the hair or textile fibre. However, there is currently no scientific explanation for this observation.

The mixtures containing (Z)-1-(3-methylbut-2-enyloxy)hex-3-ene exhibit a surprisingly high substantivity or retention towards or on hair, wool, cotton and other textile fibres.

The Examples which follow serve to illustrate the invention:

### EXAMPLES

### Preliminary Remark

In the Examples below, the properties of “substantivity” and “blooming” were evaluated by a panel of experts (8-12 persons).

For the determination of substantivity, unperfumed fabric softener, shampoo or washing powder was perfumed with the conventional concentration of (Z)-1-(3-methylbut-2-enyloxy)hex-3-ene or (Z)-1-(1-ethoxyethoxy)hex-3-ene (substance used for comparison). The olfactory assessment was made using a 7-point scale with values from 0 (no odour) to 6 (very strong odour).

For the assessment of blooming, an aqueous solution containing a low concentration of surfactant was treated with 0.1% of (Z)-1-(3-methylbut-2-enyloxy)hex-3-ene or (Z)-1-(1-ethoxyethoxy)hex-3-ene (substance used for comparison). The olfactory assessment was made using a 7-point scale with values from 0 (no odour) to 6 (very strong odour).

The “stability” property was determined on the basis of the residual amount (in %) of the test fragrances after a storage period of 1 month (1 M) or 2 months (2 M) at a constant temperature of 40° C. in the respective ready-to-use formulations (i.e. after incorporation of the respective fragrance into the appropriate base) of the following Examples.

### Determination of the Substantivity (Examples 1-3) and Stability (Examples 1 and 3)

### Example 1

### Shampoo

The substance to be evaluated was incorporated as a 50 wt. % solution in diethyl phthalate, in a dose of 0.6 wt.%, into a shampoo base of the following composition:

| | |
|---|---|
| Sodium laurylethersulfate | 12% |
| (e.g. Texapon NSO from Cognis Deutschland GmbH) | |
| Cocamidopropylbetaine | 2% |
| (e.g. Dehyton K from Cognis Deutschland GmbH) | |
| Sodium chloride | 1.4% |
| Citric acid | 1.3% |
| Rose fragrance mixture | 0.5% |
| Phenoxyethanol, methyl-, ethyl-, butyl- and propylparaben | 0.5% |
| Water | 82.3% |

The pH of the shampoo base was about 6. This is used to prepare 100 ml of a 20 wt. % aqueous shampoo solution (as an Example of a solution according to the invention). Two swatches of hair are washed together for 2 minutes in this shampoo solution and then rinsed for 20 seconds under lukewarm running water. One swatch is packed wet in aluminium foil and the second swatch is dried with a hair dryer. Both swatches are assessed by a panel for their olfactory properties.

| | Evaluation of substantivity | | |
|---|---|---|---|
| Substance to be evaluated | wet | dry | Stability |
| (Z)-1-(3-methylbut-2-enyloxy) | 2.8 | 1.2 | 1 M: 93% |
| hex-3-ene (according to the | | | 2 M: 90% |
| invention) | | | |
| (Z)-1-(1-ethoxyethoxy)hex-3-ene | 2.3 | 0.9 | 1 M: 70% |
| (comparison) | | | 2 M: 63% |

### Example 2

### Fabric Softener

The substance to be evaluated is incorporated as a 50 wt. % solution in diethyl phthalate, in a dose of 0.5 wt. %, into a fabric softener base of the following composition:

| | |
|---|---|
| Quaternary ammonium methosulfate (Esterquat), approx. 90% | 5.5% |
| (e.g. Rewoquat WE 18 from Witco Surfactants GmbH) | |
| Alkyidimethylbenzylammonium chloride, approx. 50% | 0.2% |
| (e.g. Preventol R50 from Bayer AG) | |
| Dye solution, approx. 1% | 0.3% |
| Peach fragrance mixture | 0.3% |
| Water | 93.7% |

The pH of the fabric softener base was in the range from 2 to 3. Two cloths are rinsed for 30 minutes at 20° C. with 370 g of a 1% aqueous fabric softener solution (as an Example of a solution according to the invention) in a Linetest machine running the fabric softener programme. The cloths are wrung out and then spun for 20 seconds. One cloth is sealed up wet and one is hung up to dry. Both cloths are then assessed by a panel for their olfactory properties.

Evaluation of substantivity

| Substance to be evaluated | wet | dry |
|---|---|---|
| (Z)-1-(3-methylbut-2-enyloxy) | 2.2 | 1.4 |
| hex-3-ene (according to the | | |
| invention) | | |
| (Z)-1-(1-ethoxyethoxy)hex-3-ene | 1.9 | 1.0 |
| (comparison) | | |

### Example 3

### Washing Powder

The substance to be evaluated is incorporated as a 50 wt. % solution in diethyl phthalate, in a dose of 0.4 wt. % into a washing powder base of the following formulation:

| | |
|---|---|
| Linear Na alkylbenzenesulfonate | 8.8% |
| Ethoxylated C12-18 fatty alcohol (7 EO) | 4.7% |
| Na soap | 3.2% |
| Antifoam | 3.9% |
| Dow Corning ® 2-4248S | |
| Powdered Antifoam, | |
| silicone oil on zeolite as carrier | |
| Zeolite 4A | 28.3% |
| Na carbonate | 11.6% |
| Na salt of an acrylic acid/maleic acid copolymer | 2.4% |
| (Sokalan CP5) | |
| Na silicate | 3.0% |
| Carboxymethyl cellulose | 1.2% |
| Dequest 2066 | 2.8% |
| ([[(phosphonomethyl)imino]bis[(ethylene- | 0.2% |
| nitrilo)bis(methylene)]]tetrakisphosphonic | |
| acid, sodium salt) | |
| Optical brightener | |
| Na sulphate | 6.5% |
| Protease | 0.4% |
| Sodium perborate tetrahydrate | 22.0% |
| TAED | 1.0% |

Two cloths are washed for 45 minutes at 60° C. with 370 g of a 1% aqueous washing powder liquor (as an Example of a solution according to the invention, the pH of the washing powder liquor being well in the basic range) in a Linetest machine running the main washing cycle. The cloths are first rinsed for 5 minutes with cold water, wrung out and then spun for 20 seconds. One cloth is sealed up wet and one is hung up to dry. Both cloths are then assessed by a panel for their olfactory properties.

| | Evaluation of substantivity | | |
|---|---|---|---|
| Fragrance | wet | dry | Stability |
| (Z)-1-(3-methylbut-2-enyloxy) | 2.2 | 1.8 | 1 M: 97% |
| hex-3-ene (according to the | | | 2 M: 93% |
| invention) | | | |
| (Z)-1-(1-ethoxyethoxy)hex-3-ene | 1.8 | 1.0 | 1 M: 79% |
| (comparison) | | | 2 M: 73% |

### Determination of the Blooming (Example 4)

### Example 4

The blooming of the shampoo solution of Example 1, the fabric softener solution of Example 2 and the washing powder liquor of Example 3 was assessed in each case by a panel on a scale of 0-6 from an open 250 ml glass beaker.

| Fragrance | Shampoo solution (Example 1) |
|---|---|
| (Z)-1-(3-methylbut-2-enyloxy) | 4.4 |
| hex-3-ene(according to the | |
| invention) | |
| (Z)-1-(1-ethoxyethoxy)hex-3-ene | 3.8 |
| (comparison) | |

| Fragrance | Fabric softener solution (Example 2) |
|---|---|
| (Z)-1-(3-methylbut-2-enyloxy) | 3.5 |
| hex-3-ene (according to the | |
| invention) | |
| (Z)-1-(1-ethoxyethoxy)hex-3-ene | 3.1 |
| (comparison) | |

| Fragrance | Washing powder liquor (Example 3) |
|---|---|
| (Z)-1-(3-methylbut-2-enyloxy) | 3.3 |
| hex-3-ene(according to the | |
| invention) | |
| (Z)-1-(1-ethoxyethoxy)hex-3-ene | 2.8 |
| (comparison) | |

### Fragrance Compositions

### Example 5

Fragrance composition, particularly suitable for use in shampoos, consisting of:

| | Fragrance | Fragrance |
|---|---|---|
| | mixture I | mixture II |
| | parts by | parts by |
| | weight | weight |
| Aldehyde C8, 10% in DPG | 2 | 2 |
| Aldehyde C12 lauric | 3.5 | 3.5 |
| Trans-2-hexenal, 1% in DPG | 9 | 9 |
| Cis-3-hexenol | 2 | 2 |
| Cis-3-hexenyl acetate | 4 | 4 |
| Vertocitral | 7.4 | 7.4 |
| Allylamyl glycolate | 0.5 | 0.5 |
| Dihydromyrcenol | 46 | 46 |
| Lemongrass oil | 1 | 1 |
| Geranonitrile, 10% in DPG | 2.8 | 2.8 |
| Citrylal | 0.5 | 0.5 |
| Orange oil 5x | 25 | 25 |
| Claritone ® | 5 | 5 |
| Methyl anthranilate, 10% in DPG | 1 | 1 |
| Hexyl acetate | 5 | 5 |
| Isoamyl acetate, 10% in DPG | 2 | 2 |
| Jasmaprunate | 4 | 4 |
| Prenyl acetate | 4 | 4 |
| Ethyl heptylate | 9 | 9 |
| Aldehyde C14 known as | 15 | 15 |
| Decalactone gamma | 4 | 4 |
| Ethyl 2-methylbutyrate | 5 | 5 |
| Manzanate, 10% in DPG | 4 | 4 |
| Allylcyclohexyl propionate | 3 | 3 |
| Allyl heptylate | 20 | 20 |
| Wine yeast oil green, 10% in DPG | 3 | 3 |
| Peach #D40110PM | 6 | 6 |
| Davana oil for perfumery | 0.5 | 0.5 |
| Blackcurrant #DB10002 | 6 | 6 |
| Aldehyde C16 known as | 0.5 | 0.5 |
| Lilial ® (lily of the valley fragrance) | 65 | 65 |
| Helional ® | 4 | 4 |
| Lyral ® | 6 | 6 |
| Linalool | 40 | 40 |
| Dimethylbenzylcarbinyl butyrate | 6 | 6 |
| Tagetes oil BM | 0.5 | 0.5 |
| Phenirat ® | 160 | 160 |
| Citronellol 950 | 18 | 18 |
| Geranyl acetate pure | 3 | 3 |
| Damascone delta | 1.2 | 1.2 |
| Rose booster #D50221A, 10% in DPG | 5 | 5 |
| Benzyl acetate (jasmine fragrance) | 25 | 25 |
| Alpha-hexylcinnamaldehyde (jasmine | 90 | 90 |
| fragrance) | | |
| Cis-jasmine | 3 | 3 |
| Jasmine #151 | 4 | 4 |
| Benzyl salicylate | 85 | 85 |
| Isoamyl salicylate | 30 | 30 |
| Beta-ionone | 55 | 55 |
| Irolene, 1% in DPG | 1 | 1 |
| Isoeugenol | 0.8 | 0.8 |
| Anisaldehyde | 2.5 | 2.5 |
| Vanillin | 3 | 3 |
| Cinnamon oil, 10% in DPG | 3 | 3 |
| Agrumex HC | 68 | 68 |
| Herbyl propionate | 24 | 24 |
| Vertofix BM | 80 | 80 |
| Sandolen H&R ® | 10.5 | 10.5 |
| Evernyl | 1 | 1 |
| Ambrinol S, 10% in DPG | 1.5 | 1.5 |
| Globalide ® 100% | 90 | 90 |
| Macrolide Supra, 50% in TEC | 33.3 | 33.3 |
| (Z)-1-(1-ethoxyethoxy)hex-3-ene | 80.0 | — |
| (comparative substance) | | |
| (Z)-1-(3-methylbut-2-enyloxy)hex-3-ene | — | 80.0 |
| (according to the invention) | | |

### Abbreviations Used:

DPG: dipropylene glycol; TEC: triethyl citrate

A hair washing test was carried out as indicated in Example 1 using fragrance mixture I or fragrance mixture II to perfume the shampoo.

| | Evaluation of substantivity | |
|---|---|---|
| Fragrance | wet | dry |
| Fragrance composition I | 2.3 | 1.2 |
| Fragrance composition II (according to the | 3.0 | 1.6 |
| invention) | | |

Apart from improved substantivity, the swatches washed with fragrance mixture II (according to the invention, i.e. with (Z)-1-(3-methylbut-2-enyloxy)hex-3-ene) exhibited a markedly fresher modern-green top note, which was also characterized by violet, lily of the valley, jasmine and apple aspects.

1-10. (canceled)

11. A process for increasing the substantivity and/or retention of a fragrance mixture comprising adding (Z)-1-(3-methylbut-2-enyloxy)hex-3-ene to a fragrance mixture.

12. A process for providing hair or textile fibres with a fresh-green smell, comprising:
(1) preparing a mixture comprising (Z)-1-(3-methylbut-2-enyloxy)hex-3-ene; and
(2) applying said mixture comprising (Z)-1-(3-methylbut-2-enyloxy)hex-3-ene to said hair or textile fibres.

13. The process of claim 12, wherein said mixture comprising (Z)-1-(3-methylbut-2-enyloxy)hex-3-ene is a solution comprising (a) water, (b) (Z)-1-(3-methylbut-2-enyloxy)hex-3-ene, and (c) one or more surfactants; wherein the concentration of (b) in said solution is in the range of from 10⁻⁷ to 10⁻¹ weight %.

14. A solution comprising (a) water, (b) (Z)-1-(3-methylbut-2-enyloxy)hex-3-ene, and (c) one or more surfactants; wherein the concentration of (b) in said solution is in the range of from 10⁻⁷ to 10⁻¹ weight %.

15. The solution of claim 14, wherein the pH of said solution is in the range of from 8 to 13.

16. A process for increasing the odour of other fragrances perceived above an aqueous solution containing surfactants comprising adding (Z)-1-(3-methylbut-2-enyloxy)hex-3-ene to an aqueous solution containing surfactants.

17. A mixture comprising (Z)-1-(3-methylbut-2-enyloxy)hex-3-ene and one or more surfactants selected from the group consisting of linear alkylbenzenesulfonates, fatty alcohol ethoxylates having 12 to 18 C atoms, laurylethersulfates, and betaines.

18. The mixture of claim 17, wherein said linear alkylbenzenesulfonates are present in a proportion in the range of from 7 to 10 weight % based on the total weight of the mixture and/or said fatty alcohol ethoxylates having 12 to 18 C atoms are present in a proportion in the range of from 3 to 6 weight % based on the total weight of the mixture.

19. The mixture of claim 17, wherein said laurylethersulfate is sodium laurylethersulfate and wherein said sodium laurylethersulfate is present in a proportion in the range of from 7 to 13 weight % based on the total weight of the mixture and/or said betaines are present in a proportion in the range of from 1 to 3 weight % based on the total weight of the mixture.
